(19) United States
(12) Patent Application Publication
(10) Pub. No.: US 2008/0125727 A1
(43) Pub. Date: May 29, 2008

(54) APPARATUS FOR THE ADMINISTRATION OF A FLUID PRODUCT

(76) Inventors: Alexander Seibold, Liederbach (DE); Fritz Kirchhofer, Sumiswald (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/847,032

(22) Filed: Aug. 29, 2007

(30) Foreign Application Priority Data
Aug. 30, 2006 (DE) DE 102006040678.8

(51) Int. Cl. A61M 25/00

(52) U.S. Cl. 604/264

(57) ABSTRACT

An apparatus for the administration of a medicine, the apparatus including a hydraulic passage and a porous element, in some embodiments, a membrane, in the hydraulic passage, wherein the rate of administration is controlled, adjusted or selected by determining the rate of flow or delivering a component of an impulse pressure of a hydraulic fluid flowing through the passage and porous element. In some embodiments, different porous elements, a porous element having portions of varied permeability or a cover for covering all or a portion of the porous element may be used for selectively controlling or varying the rate of flow and, thereby, the rate of administration of the medicine.

### CROSS-REFERENCE TO RELATED APPLICATIONS

This application claims benefit of priority to German Application No. DE 10 2006 040 678.8 filed on Aug. 30, 2006, the content of which is incorporated in its entirety herein by reference.

### BACKGROUND

The present invention relates to devices for injecting, infringing, administering, delivering and dispensing substances, and to methods for making and using such devices. More particularly, the present invention relates to apparatus or devices for the administration of a fluid product or substance, wherein hydraulic forces are involved or utilized in the administration of the product or substance. More particularly, the present invention relates to devices for administering a fluid product, e.g., a medicinal substance, comprising a hydraulic reservoir containing a hydraulic fluid, a drive element for generating an impulse pressure on the hydraulic reservoir, a fluid connection between the hydraulic reservoir and a fluid receptacle through which the hydraulic fluid can flow, and a product container containing the fluid product, wherein hydraulic fluid which reaches the fluid receptacle causes the fluid product to be forced out of the product container. Such apparatus and devices are well suited to the administration by infusion of a medicine in liquid form.

In the case of many illnesses it is desirable to administer medicine or a medicinal substance or product, e.g. insulin preparation or a blood-thinning medicine like Heparin, to a patient continually in a fluid form. Infusion devices, wherein the medicine is present in a glass ampoule, are known for this purpose. Generally, a catheter links an ampoule to a cannula, which discharges into the patient's body tissue, e.g. subcutaneously. There is a movable stopper in or on the ampoule. The medicine is dispensed to the patient through the catheter and the cannula by moving the stopper.

In the prior art, infusion devices are disclosed and suggested in which the stopper is not moved directly, but rather by a hydraulic system. In this way, measurement of the rate of the medicine's administration took place by the hydraulics. Impulse pressure on a hydraulic reservoir is generated in an appropriate way, e.g. by a pressure spring. The escaping hydraulic fluid travels or reaches over or through a hydraulic passage to the ampoule and there pushes the product stopper forward. Typically, there is a device for limiting rate of flow in or through the hydraulic passage to ensure a constant, low rate of flow through the hydraulic passage and, thereby, an appropriate rate of administration of the medicine.

An example of such an apparatus is described in DE-A 199 39 023. In the hydraulic passage, a long capillary of small cross-section is envisaged, which serves in limiting rate of flow and reducing pressure.

A disadvantage of this kind of apparatus is that the capillary passage must be very precisely crafted to avoid excessive variations in the rate of administration within a product load. Generally, the design of such a passage is governed by the Hagen-Poiseuille law, which states the relationship between the rate of laminar flow through a cylindrical capillary and the dimensions of the capillary. According to this law, the fourth power of the capillary's diameter is proportional to the calculation of the rate of flow. For this reason, small variations in the cross section of the capillary can themselves lead to large variations in the rate of flow. Since the capillaries demand extremely precise fabrication, the manufacturing costs are relatively high. These kinds of apparatus are, therefore, typically suitable for single use. In addition, the danger exists that even the smallest impurities in the hydraulic system may lead to a blockage in the capillary, so that the rate of flow through the capillary is interrupted and no medicine is administered. This can lead to a dangerous, often even life-threatening, undersupply of medicine.

Also, with one capillary passage it is only possible to administer the medicine at a single, predetermined, constant rate, which is determined by the length and diameter of the capillary passage. Therefore, no control of the rate of administration is possible. However, in practice it is often necessary to change the rate of administration to tune or adjust it to the individual needs of a patient.

### SUMMARY

It is an object of the present invention to provide an apparatus for the administration of a fluid product of the type described above, with which measurement and/or control of the rate of administration of a product or substance can be carried out in a simple and cost-efficient way.

In one embodiment, the present invention comprises an apparatus or device for the administration of a medicine, the apparatus comprising a hydraulic passage and a porous element, in some embodiments, a membrane, in the hydraulic passage, wherein the rate of administration is controlled, adjusted or selected by determining the rate of flow or delivering a component of an impulse pressure of a hydraulic fluid flowing through the passage and porous element. In some embodiments, different porous elements, a porous element having portions of varied permeability or a cover for covering all or a portion of the porous element may be used for selectively controlling or varying the rate of flow and, thereby, the rate of administration of the medicine.

In one embodiment, administration apparatus or device for administering a fluid product according to the present invention comprises: a hydraulic reservoir with hydraulic fluid, a drive element for the generation of impulse pressure on the hydraulic reservoir, a fluid connection between the hydraulic reservoir and a fluid receptacle, a product container with the fluid product, which is fashioned in such a way that hydraulic fluid which reaches the fluid receptacle effects a discharge of the fluid product out of the product container, and a porous element housed in the fluid connection which is permeable to or permits the passage of the hydraulic fluid to measure and/or control the rate of flow. The rate of administration is therefore measured and/or controlled within or by the hydraulic channel or flow or movement of the hydraulic fluid through the fluid connection and the associated porous element.

Thus, in some embodiments, the porous element determines the rate of flow and, thereby, the rate of administration as well. A porous element easily allows for controlled passage of the hydraulic fluid at a predetermined rate of flow without necessitating complex mechanical elements such as mechanically crafted capillary passages with a cross section defined for rate of flow. As a rule, there are a multitude of fine spaces, opening or channels within a porous element which enable the passage of hydraulic fluid, and these channels could be separate individual channels or could build or form a widely ramified network. The permeability of a porous element can be set easily, within narrow tolerances, as far as production is concerned. Finally, the flow rate through a porous element is determined by the average characteristics of a very large number of channels, which is how variations in the characteristics of individual channels are balanced out.

Various kinds of porous elements are known. For example, a porous element can be built or formed by a porous ceramic block or a sponge, which may be introduced into the fluid connection. In some preferred embodiments, the porous element at least contains or comprises a membrane. Membrane should be understood as meaning a thin, porous element, i.e. a porous element which exhibits a smaller or much smaller measurement in one spatial dimension than in others, e.g., the other two. A membrane is usually thinner than one millimeter, often thinner than 200 micrometers. Diverse porous materials with differing porosities are known, such as near-micro-porous materials with a pore size smaller than 2 nm or macro-porous materials with a pore size over 50 nm. The choice of porous material in embodiments of the present invention may be made in accordance with the desired rate of administration of a fluid medicine and, therefore, with the required rate of flow through the fluid connection on the administration device.

In some embodiments, to regulate the rate of flow, the porous element can also be partly covered, so that the hydraulic fluid flows through only a selected portion of the porous element. In some embodiments, and in some embodiments at the same time a partial cover is used, the permeable portion can be adjustable or selectable in size, to change the rate of flow or to interrupt the flow altogether. For example, an adjustable aperture can be laid over or adjacent to the membrane and can then serve as selection element for choosing the size of the permeable portion and/or the blocked, sealed or occluded portion.

In some preferred embodiments, a screen with a plurality and/or selection or pattern of openings of definite size may be arranged over or adjacent to the porous element. The screen and/or the opening thereof can be brought or moved into a selected or a selection of defined positions. Dependent on the position selected, openings on the screen then unblock variably permeable portions, and/or portions of varying size, of the porous element. Alternatively, it can also be arranged that there is a position such that the screen totally covers the porous element to completely interrupt the flow. In this way, the porous element can be held stationary within the administration apparatus casing while the screen is adjustable relative to the porous element, or the porous element can be attached to the screen and moveable with it, such that in every position of the screen another area of the porous element is permeated. Also, different porous elements can be attached over the individual openings on the screen, such that in every position of the screen another porous element is permeated. In this case, the rate of flow though the hydraulic passage can be adjusted by choosing one of the several different porous element, whereby the size of the area of porous section can remain constant.

In some embodiments, the choice of rate of flow, e.g. as established by the size of the permeable area or permeable section of a porous element or by using a selected porous element, can be adjusted manually before or during administration. However, it is also possible to attach a set of automatic controls to the selection element, through which the rate of administration can be changed automatically with time. Such controls can be responsive to changing needs in terms of quantity of medicine needed e.g., in a treatment, in a day, during a period of administration, during the day, etc.

In some embodiments of the present invention, the screen is a compact, basically tubular design, i.e. it exhibits at least one area with a circular cylindrical surface, in which openings are arranged. Thus, the screen can be brought to the majority of positions by turning the cylinder axis. In this case, the porous element may be arranged fixed near the wall of a guidance element (which also may be thought of and/or referred to as a guide, guide element, etc.) complementary to the screen, i.e. a guidance element at least partly cylindrical, and exhibiting the shape of an elongated strip running parallel to the cylinder axis. In some preferred embodiments, the surface of the strip facing the screen is curved around the cylinder axis relative to its radius. It can, however, be generally flat, so long as the dimensions of the strip tangential to the direction of curvature are not too large.

In some preferred embodiments, strip-like sealing elements are arranged between the openings of the screen, which span or extend parallel to the cylinder axis on the surface of the screen, protruding out of the surface of the screen and fit pressure-tight into the guidance element on both sides of the porous element. In this way, a tangential leakage of hydraulic fluid between the screen and the guidance element can be reduced or prevented.

In some embodiments, to achieve definite positions of the screen relative to the guidance element, at least one cavity can be drilled in the guidance element, with which at least one of the sealing elements engages. In some preferred embodiments, there is a complementary cavity in the form of an elongated groove arranged for each of the sealing elements.

In some embodiments, the roles of the seals and the cavities can be reversed, i.e. sealing elements can be arranged on the guidance element instead of on the screen, and cavities, e.g. elongated grooves parallel to the cylinder axis, can be drilled into the screen with which the sealing elements of the guidance element can engage. This prevents the sealing elements from sliding along the screen and thereby damaging it while the screen is being twisted.

In some preferred embodiments, there are graphics, e.g. a scale, on the administration apparatus on or by which the position of the selection element, such as the screen, or the choice of porous element can be displayed or indicated. A dedicated element for display on the scale, or the scale itself, is thereby linked with the selection element, and/or with the screen.

In some preferred embodiments, the screen surrounds the product container at a predetermined distance, such that between the screen and the product container an annular space emerges. This then forms part of the fluid receptacle.

Various types of product containers can be installed in the apparatus. The product container may be a commercially available ampoule, i.e. an ampoule with a cylindrical sidewall and a product stopper which can be pushed into it. The ampoule is then arranged in such a way that hydraulic fluid flowing through the porous element and reaching the fluid receptacle moves the product stopper in the direction of the product container outlet so that the fluid product is administered. Instead of this, the product container can also be a flexible pouch or another repository with a deformable outer wall, so that the product container can be compressed by the hydraulic fluid coming through the porous element. An example is a container with a bellows-like and/or deformable wall portion or wall, i.e. a wall with a variety of bending lines, along the length of which neighbouring sidewall areas fold one upon another.

In some embodiments of the present invention, the hydraulic reservoir can be delimited by a moveable stopper or it can be compressible in its entirety.

The apparatus can be powered in various ways in various embodiments. In one reliable and cost-efficient variant, the drive element comprises a coil spring which generates a force on the hydraulic reservoir. Motors, like conventional multiphase motors, are also suitable drives.

In some embodiments, by using a selectively permeable or semi-permeable porous element, e.g. a type of membrane or diaphragm, the force of an osmotic effect can be used as a drive or driving force for the administration of a fluid medicine from the administration apparatus. If fluids with differing concentrations of a solvent are introduced on either side of the membrane, a drive results from the diffusion of the solvent from one side of the membrane to the other through the selectively porous element. For example, a fluid can be provided in the hydraulic reservoir with one concentration of a solvent and in the fluid receptacle with a second concentration, wherein the hydraulic reservoir is separated from the fluid receptacle by a selectively porous element. A combination of drives is also conceivable, such as a spring and a pressure generation apparatus based on the osmotic effect.

In some embodiments, the apparatus and methods of the present invention are well-suited for the administration of medicines such as antibiotics, insulin preparations, blood-thinning medicines, growth hormones, etc.

### BRIEF DESCRIPTION OF THE DRAWINGS

FIG. 1 is a cross-section of one embodiment of an apparatus according to the present invention;

FIG. 2 is a schematic representation of the true length of a screen in accordance with the present invention;

FIG. 3 is a cross-section of an ampoule with a screen encircling it;

FIG. 4 is a plan view of the apparatus of FIG. 1; and

FIG. 5 is a cross-section through the apparatus of FIG. 1 along the line V-V.

### DETAILED DESCRIPTION

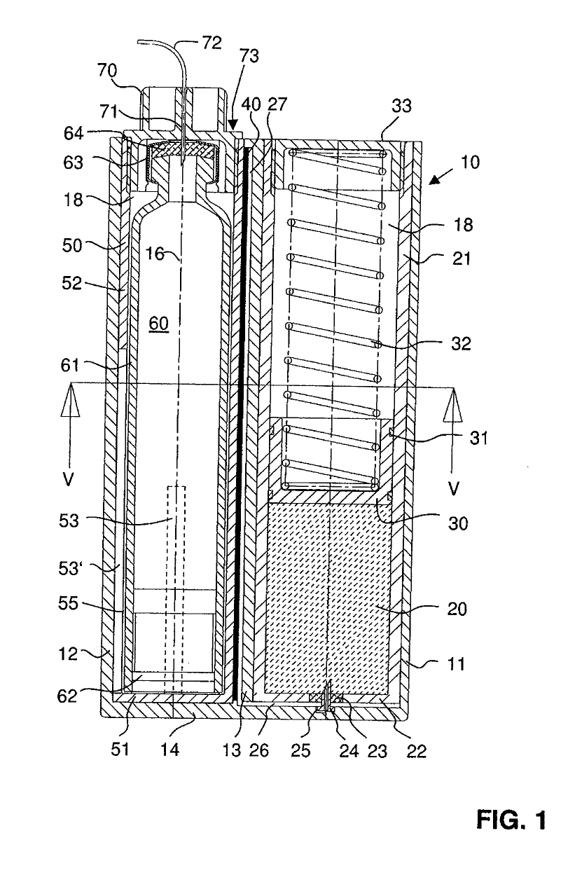

An exemplary embodiment of an apparatus or device in accordance with the present invention is depicted in FIGS. 1 to 5 in various views.

As is noticeable in FIGS. 1 and 5, the apparatus is comprised of a casing 10 with two parallel, hollow, cylindrical casing components 11 and 12, which are connected to each other in the area of the cylindrical sidewalls over a connecting wall 13. Extra stability is lent to the casing by a common casing bottom 14 as well as by a common casing top 15, as shown in FIG. 4.

A hydraulic reservoir 20 and a drive with a coil spring 32 are located in a cylindrical hollow space 17 in the first casing component 11, in FIGS. 1, 4 and 5, on the right hand side. A product container in the form of a medicine ampoule 60 is incorporated in a hollow space 18 running parallel to this within the second casing component 12, in FIGS. 1, 4 and 5, on the left hand side.

The cylindrical hydraulic reservoir 20 in the hollow space of the first casing component 11 is delimited laterally by a circumferential cylindrical sidewall 21 and underneath or on one end by a bottom 22. The hydraulic reservoir is delimited by a hydraulic stopper 30 which is moveable in the direction of the cylinder axis and which is sealed by sealing rings 31 against the sidewall 21.

There is a mainspring 32 working as a pressure spring in the form of a coil spring located adjacent to the hydraulic stopper 30. This mainspring serves as drive for the administration of the medicine. The spring is compressed between the hydraulic stopper 30 and a top cover 33 which is screwed into the encompassing sidewall 21 of the hydraulic reservoir 20, and so it produces a driving force working on the hydraulic stopper, which leads to an increase in pressure in hydraulic reservoir 20. It is possible to additionally provide a retainer for the spring drive which interrupts the power transmission to the hydraulic fluid. By releasing the retainer the power flow to the fluid medicine can be started. An interlock can be provided as a retainer, for example.

A seal 23 is built into the bottom 22 of the hydraulic reservoir 20. (It should be understood that positional, directional and/or relative terms such as bottom, top, left, right, etc. are, unless otherwise stated, used for convenience, not as limitations.) A hollow needle 24 is inserted into this seal. The lower end of the hollow needle 24 ends in a fluid channel 25, which leads into a gap or space 26 between the casing 10 and the bottom 22 of the hydraulic reservoir 20. A fluid chamber 27 is connected to the space 26, is built into the connecting wall 13 along its length and extends parallel to the cylinder axis 16 of the second casing component 12. This elongated continuing fluid chamber 27 is recognizable in FIG. 5. There is, therefore, a fluid connection between the hydraulic reservoir 20 and the fluid chamber 27. The cross section of this fluid connection is overall sufficiently large for the fluid connection not to fundamentally hinder the flow of the hydraulic fluid. In other words, the impulse pressure in the hydraulic reservoir 20 is transferred across the fluid connection to the fluid chamber 27 with only little or no fall in pressure.

The fluid chamber 27 is separated from the second (left) casing component 12 by a membrane 40, which allows a controlled, slow movement of hydraulic fluid in the direction of the second casing component 12.

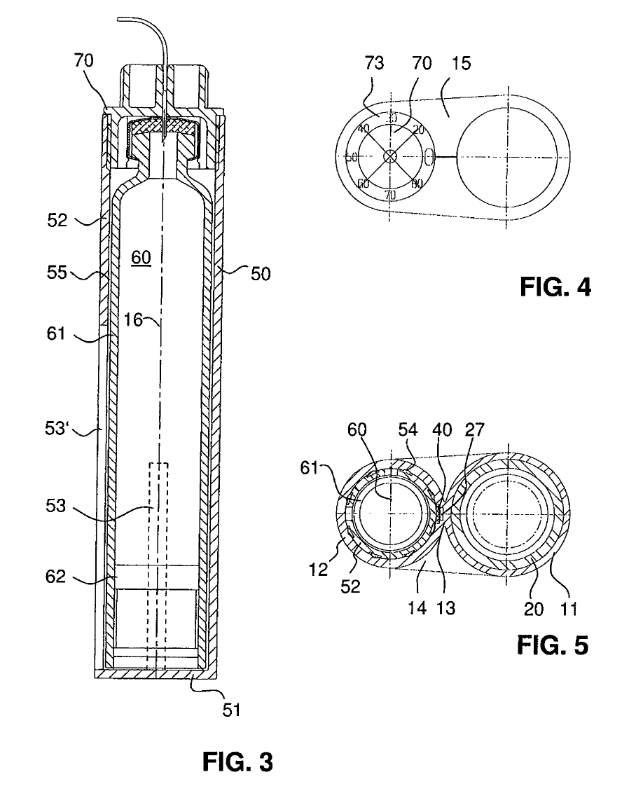

The membrane 40 is in part or totally covered on the outlet side by a screen 50 in the form of a cylindrical covering with sides walls 52 and bottom 51. The screen 50 is in the elongated cylindrical hollow space 18 in the left casing component 12. The screen 50 surrounds the medicine ampoule 60, as is recognizable from FIG. 3, in which the casing 10 is not shown. The medicine ampoule 60 features an encircling sidewall 61 open to the bottom, as well as a product stopper 62 which can be pushed into it. These kinds of medicine ampoules are widespread on the market and available in various sizes. In a prevalent form, an ampoule contains a volume of e.g. 3 milliliters of fluid medicine.

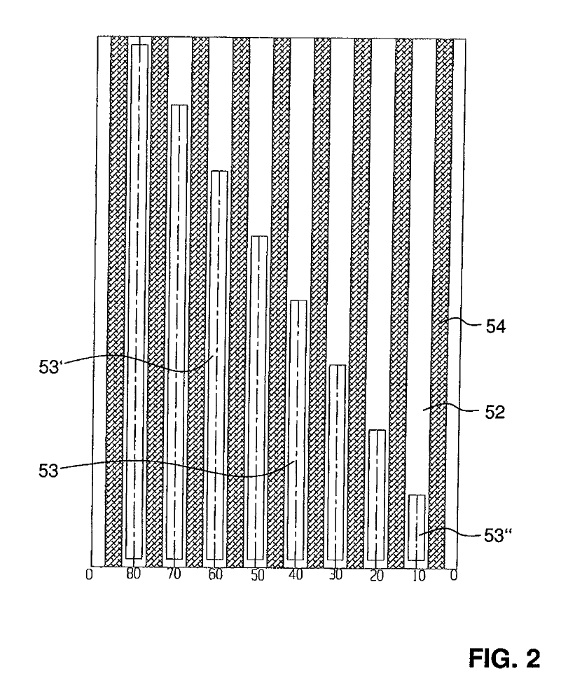

A true length version of the screen 50 is shown schematically in FIG. 2. Formed in the sidewall 52 of the screen 50 there are several (here eight) elongated lamellar openings or areas 53, 53′, 53″, etc. of varying sizes running parallel to the cylinder axis 16. Stretching between the openings and parallel to their length there are strip-like, elongated seals 54, which are fixed into corresponding flat grooves in the screen 50. The seals may be made from, for example, a suitable elastomer. As can be seen from FIG. 5, every seal 54 with a rounded area overlaps the outer side of the sidewall a little. There are corresponding nuts built into the inner side of the second casing component's sidewall 12, into which the seals 54 can engage. The screen 50 can be turned about the cylinder axis 16 between a variety of positions, in each of which the seals 54 sit or lodge in the grooves. A certain force must therefore be overcome in order to deform the seals sufficiently that they leave the grooves and can slide along the inner side of the casing. Conversely, the positions in which the seals are once again engaged in the grooves are noticeable in and of themselves. In this way the screen 50 can be exactly positioned by turning it about the cylinder axis 16.

In every position, the screen 50 abuts or is against the membrane 40 forming a seal. An opening 53, 53′, 53″, etc. leaves, in each case, an area of the membrane of differing size free. In contrast, outside each opening the sidewall 52 of the screen 50 covers the membrane 40 in such a way that hydraulic fluid can only go though that membrane area which is uncovered because of the opening. In this way, the rate (volume per unit time) at which hydraulic fluid goes through the membrane can be set. A measurement of this is given on the lower scale of FIG. 2 (in arbitrary units). In another position (on the lower scale of FIG. 2 this is position “0”), the screen totally covers the membrane.

Between the screen 50 and the medicine ampoule 60 there is a narrow annular space 55, which gets larger at the upper end of the ampoule. Hydraulic fluid flowing through the membrane 40 and the screen 50 enters this annular space. At the top, the annular space 55 is delimited by a fastener 70, which is screwed into the screen 50. The ampoule 60 can therefore be turned together with the screen 50 located in the casing 10. In addition, within the fastener 70 a hollow needle 71 is held, the lower end of which is led through a septum 64 in the ampoule lid 63 and thereby forms an exemplary opening in the ampoule. The upper end of a hollow needle 71 is additionally connected to a catheter 72. At the farthest end of the catheter 72 there is affixed, as a rule, a cannula (not shown) for infusion into the patient's body. There is a scale 73 available on the upper side of the fastener 70 showing the rotational position of the fastener and, with it, that of the screen 50. A measurement of the rate of administration currently set is thereby shown on the scale.

Before starting the apparatus, i.e. when it is delivered to the patient, the hollow needle 71 has not yet been completely pushed through the septum 64, or the outflow of medicine is otherwise blocked. In addition, the screen is at position “0”. The mainspring 32 is compressed in an initial position. The hydraulic reservoir 20, the fluid chamber 27, the fluid connection positioned between them as well as the annular space 55 are totally filled with hydraulic fluid, and the membrane 40 is correspondingly bathed in hydraulic fluid on both sides. The apparatus is then brought into operation by sticking the hollow needle 71 completely though the septum 64 or otherwise enabling an outflow of medicine into the catheter 72. An excess pressure initially prevailing in the ampoule 60 thereby leads to a small volume of medicine being forced out though the hollow needle 71 and the catheter 72 to eliminate the air there present or, in other words, to prime the device. In the operation of the apparatus, the mainspring 32 then pushes the hydraulic stopper 30 downwards. Hydraulic fluid from the hydraulic reservoir 20 is pressed into the fluid chamber 27, in which a corresponding pressure equally prevails. Because of this pressure, hydraulic fluid is pressed through that area of the membrane 40 which is left uncovered by the openings 53, 53′, 53″, etc. in the screen 50 situated above it. This happens at a relatively low rate, whereupon this rate can be set by the size of the opening in the screen 50. The hydraulic fluid coming through the screen reaches the annular space 55. It exerts a force on the product stopper 62 by which the medicine is forced through the hollow needle 71 and the catheter 72 out of the ampoule 60. The medicine's rate of administration thereby corresponds to the rate of flow of the hydraulic fluid through the membrane.

Suitable membranes are known in the prior art. In preferred embodiments of the present invention, membrane 40 may be chosen on the basis of various criteria, e.g. with regard to the desired rate of administration, to the hydraulic fluid being used and to the impulse pressure. De-ionised or distilled water may be used as hydraulic fluid, and, as the need arises, may be combined in small concentrations with a suitable dye and put to use for visually checking the fill level of the hydraulic reservoir, which, in some embodiments, may be refillable. Water is suitable due to its physiological innocuousness, but also because a very large number of membranes have been developed for it. These kinds of membranes are normally adopted for, e.g., water treatment in desalination equipment (reverse osmosis), for dialysis (haemofiltration), in batteries and fuels, in dairies, in chemical separation technology and analysis, etc. Known organic and inorganic materials include, e.g., cellulose acetate, interlaced fibreglass yarns, ceramics (e.g., made of ATZ=alumina, titania, zirconia), mixed nitrocellulose esters (MCE), polyamide/nylon, polycarbonate (PCTE), polyester sulphone (PES), polyester (PETE), polyimide, polypropylene or polytetrafuoroethylene (PTFE). It is also possible to superimpose the membrane upon a suitable (as a general rule, porous) support. Multilayered membranes are also possible. This kind of membrane composite can contain several thinner membranes made of the same or of differing materials. For example, so-called Thin Film Composite Membranes (TCM or TFM) are known from the field of water preparation, and are comprised of, e.g., a polyimide membrane on a porous polysulphone support.

In accordance with the present invention, by choosing a suitable hydraulic fluid in combination with a suitable membrane, a very large range of rates of flow can be set up, which can range from a quick administration of the entire contents of an ampoule over a period of a few seconds to a very slow administration over a period of days or even weeks.

An example of a membrane suitable for use in accordance with the present invention, would be a typical polycarbonate membrane with an average pore size of 0.03 micrometers at a pore density of 6×10⁸ cm⁻², across which a pressure differential of 0.7 bar would exhibit a rate of flow of around 0.2 ml min⁻¹ cm⁻². On an active (uncovered by the screen) membrane area of 2 cm×2 mm=0.4 cm² a rate of flow of 0.08 ml per minute results, i.e. a relatively quick administration of an ampoule of 3.0 ml over a time period of just under 40 minutes. By use of a composite of several such membranes, of membranes made of another material, of another thickness, pore density and/or pore size, etc. larger or smaller rates of flow can be achieved. For the administration of medicines like insulin or blood-thinning medicines the rate can amount to 0.1 to 10 millilitres per day.

Instead of a coil spring, a spiral spring could be used, for instance, or a drive such as is known from mechanically powered clockwork. The drive also does not need to be effected by a spring, but can also be effected in other ways, e.g. electromotively. The effect of the drive can additionally be strengthened or weakened by osmotic effects. For example, in the fluid space 27 there can prevail a higher concentration of dissolved substances unable to permeate the membrane than in the annular space 55, such that the osmotic pressure works against the fluid pressure generated by the driving power, or vice versa.

A variety of configurations are possible for the apparatus of the present invention, comprising optional relative adjustments and alignments of the hydraulic reservoir relative to the medicine ampoule. The hydraulic reservoir can be located e.g. underneath the medicine ampoule along the length of the cylinder axis 16. The hydraulic reservoir can also be constructed as an annular space, as is for example illustrated in the DE-A 199 39 023. In this case the annular hydraulic reservoir encircles the medicine ampoule, the screen and the membrane.

Instead of a membrane in the literal sense, a different porous element of suitable form could be used, e.g. a rod-shaped element made of a suitable porous ceramic or a corresponding element containing zeolite.

The medicine container can be available in optional sizes. Instead of a medicine ampoule, a different kind of medicine container can also be adopted, e.g. a flexible and/or elastic medicine pouch or a container with a bellows-like wall. The same is true for the hydraulic reservoir.

In the exemplary embodiment described herein, the medicine container 60 is encircled by the screen 50 and can be turned with it. Instead of this, the medicine container could be held fast in the casing 10, and the screen could be moved relative to the medicine container. A membrane could also be attached directly to the screen, e.g. affixed to the screen only in the area of the openings or all around within and/or outside. In this case, the fluid space 27 could be open to the screen or delimited by an extra membrane. If a membrane is attached to the screen, every opening on the screen may be allocated to a different kind of membrane. In this way it is then possible to achieve different rates of flow even if all the opening on the screen are of the same size.

While in the exemplary embodiment described herein, exactly one permeable area of the membrane is uncovered at every position of the screen, it could also be the case that several openings, separate one from the other, could be provided, which together uncover the permeable area. In this way, the stability of the screen can or could be changed. This is advantageous if a membrane is attached directly to the screen.

Other arrangements for a selection element are also conceivable, depending on the configuration. For example, the selection element can be constructed as a flat plate arranged over the membrane so as to be moveable. With this kind of sliding controller the portion of the porous element selected for flow can also be continually changed advantageously. For this, the sliding controller is pushed along the length of the membrane surface and partially covers and/or seals it. Several other configurations of a selection element are conceivable.

In some embodiments, the screen can be altogether discarded, and thus a device or apparatus exhibiting or providing a sole or single predetermined rate of flow is made possible.

Embodiments of the present invention, including preferred embodiments, have been presented for the purpose of illustration and description. They are not intended to be exhaustive or to limit the invention to the precise forms and steps disclosed. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A device for administering a substance, the device comprising a hydraulic passage and a porous element associated with the hydraulic passage, wherein the porous element affects a hydraulic fluid in the passage, thereby controlling the rate of administering.

2. The device according to claim 1, wherein an affect of the porous element is on a rate of flow of the hydraulic fluid, the rate of flow selectively controlled by one of a selected porous element or a porous element having portions of varied permeability.

3. The device according to claim 2, wherein the porous element comprises a membrane.

4. The device according to claim 1, whereby the porous element is covered such that a selected portion of the porous element is permeable to the hydraulic fluid.

5. The device according to claim 4, whereby the selected portion can be altered by a selection element to change the rate of flow.

6. The device according to claim 1, wherein the porous element comprises permeable portions of different sizes, the device further comprising a selection element comprising a screen with a plurality of openings, wherein the screen is generally adjacent to the porous element and is moveable into a variety of selected positions whereby, depending on the selected position, selected openings of the screen are aligned with a selected permeable portion or permeable portions.

7. An administration apparatus for the administration of a fluid product, comprising:
a hydraulic reservoir containing a hydraulic fluid;
a drive element for generating an impulse pressure on the hydraulic reservoir;
a fluid connection between the hydraulic reservoir and fluid receptacle;
a product container containing the fluid product, wherein hydraulic fluid reaching the fluid receptacle causes the fluid product to be forced out of the product container; and
a porous element in the fluid connection, said porous element permeable to the hydraulic fluid and affecting a rate of flow through the fluid connection.

8. The administration apparatus according to claim 7, whereby the porous element comprises a membrane.

9. The administration apparatus according to claim 7, whereby the porous element is covered such that a selected portion of the porous element is permeable to the hydraulic fluid.

10. The administration apparatus according to claim 9, whereby the permeable portion can be altered by a selection element to change the rate of flow.

11. The administration apparatus according to claim 7, wherein the porous element comprises permeable portions of varied size, said apparatus further comprising a selection element comprising a screen with a multitude of openings generally adjacent to the porous element, wherein the screen can be moved to a multitude of positions whereby, depending on the position, selected openings of the screen uncover a selected permeable portion or selected permeable portions of the porous element.

12. The administration apparatus according to claim 11, whereby the screen comprises a circular cylindrical surface having a cylinder axis, the openings in said surface, and wherein the screen can be moved by turning the screen about the cylinder axis.

13. The administration according to claim 12, further comprising an at least partly cylindrical guidance element having a wall, wherein the porous element is located in the wall and comprises an elongated strip parallel to the cylinder axis.

14. The administration apparatus according to claim 11, wherein the screen at least partly surrounds the product container, and wherein the fluid receptacle comprises an annular space between the screen and the product container.

15. The administration apparatus according to claim 7, whereby the product container comprises an ampoule with a cylindrical sidewall and a product stopper which can be pushed into the ampoule, wherein the ampoule and stopper are arranged such that hydraulic fluid entering the fluid receptacle pushes on the product stopper to administer the fluid product.

16. The administration apparatus according to claim 7, further comprising a moveable selection element comprising at least two porous elements of differing porosities, wherein a selected one of the porous elements can be moved into the fluid connection by moving the selection element.

17. The administration apparatus according to claim 16, wherein the selection element comprises a circular cylindrical surface having a cylinder axis, the at least two porous elements being associated with the surface, whereby the selection element can be turned about the cylinder axis to be moved into a position in which only one of the porous elements is in the fluid connection.

18. The administration apparatus according to claim 10, wherein the selection element is moveable over a surface of a porous element.

19. The administration apparatus according to claim 18, further comprising a scale arranged to display the position of the selection element.

20. An administration apparatus for the administration of a fluid product, comprising:
a hydraulic reservoir containing a hydraulic fluid;
a drive for generating an impulse pressure on the hydraulic reservoir, said drive comprising at least one power module generated by osmotic pressure;
a fluid connection between the hydraulic reservoir and a fluid receptacle;
a product container containing the fluid product, wherein hydraulic fluid reaching the fluid receptacle causes the fluid product to be forced out of the product container.

21. The administration apparatus according to claim 20, further comprising a selectively porous element wherein fluids with different concentrations of a solvent are present on either side of the element.

22. The administration apparatus according to claim 21, wherein the drive further comprises another power module comprising a drive element, wherein the osmotic pressure power module supports or works against the another power module.

23. The administration apparatus according to claim 20, wherein the drive further comprises another power module comprising a drive element, wherein power module generated by osmotic pressure supports or works against the another power module.
